(19) United States
(12) Patent Application Publication
(10) Pub. No.: US 2024/0003910 A1
(43) Pub. Date: Jan. 4, 2024

(54) METHODS FOR USE IN PREVENTATIVE HEALTHCARE

(71) Applicant: RANDOX LABORATORIES LTD, Crumlin Antrim (GB)

(72) Inventors: Paul Innocenzi, Crumlin Antrim (GB); Peter Fitzgerald, Crumlin Antrim (GB); McMaster Steven, Crumlin Antrim (GB)

(21) Appl. No.: 18/037,968

(22) PCT Filed: Nov. 15, 2021
(86) PCT No.: PCT/EP2021/081688

§ 371 Date: May 19, 2023

(30) Foreign Application Priority Data
Nov. 20, 2020 (GB) 2018286.1

(51) Int. Cl. G01N 33/68

(52) U.S. Cl. CPC G01N 33/6893; G01N 2800/7042

(57) ABSTRACT

Methods and products for adjusting the chronological age of a biological system or an individual to provide finer scale insight to health status are provided. The methods are underpinned by various serum and urinary biomarkers including antistreptolysin.

### FIELD OF THE INVENTION

The invention relates to a method for adjusting the chronological age of a biological system or an individual to provide finer scale insight to health status. This enables intelligible and rapid implementation of measures to optimise an individual's health.

### BACKGROUND

Public healthcare systems are facing a dual existential threat globally due to a growing and ageing global population and a real-terms decrease in financial funding. An ageing population increases the number of individuals with chronic and age-related medical conditions such as Alzheimer's disease, heart disease and cancer. To compound the financial burden, most of these conditions lack a cure and are managed using hands-on healthcare support and pharmaceutical drugs. Furthermore, traditional healthcare methods are reactive, the individuals visiting a clinic when already diseased. This sub-optimal healthcare provision model is being further undermined by the on-going decrease in production of novel, innovative drugs by pharmaceutical companies. A modern approach to patient welfare is the implementation of preventative healthcare driven by improved diagnostic systems with the aim of maintaining a disease-free life or increased ‘healthspan’ through the identification of early-warning markers of pre-disease and disease. Consumers are aware of the short-comings of the current healthcare model and, wishing to take greater control of their own well-being and to pursue a disease prevention approach, have driven the proliferation of direct-to-consumer diagnostic testing services. These testing services can be broadly partitioned into imaging services, such as X-ray, ultrasound, CAT scan, MRI, and biological sample testing services in which a sample from the patient, usually blood or urine, is analysed for specific components. Most consumer testing services provide a limited menu of well-established analytes which can be tested.

Personal fitness and nutrition awareness complement the preventative healthcare model. However, these discrete services are often pursued on an uncoordinated and individual basis. Biological sample testing is the cheapest and most practical approach to a preventative healthcare model but its implementation faces several standardisation challenges. Laboratory practice standards exist to control variability arising through the use of different test reagents, test methods and protocols, sample analysers, sample handling and other methods, albeit generally of a smaller magnitude, the intra-analyser/reagent variability. However, significant variability still exists between and within different test providers. These disparities can lead to different clinical interpretation and ultimately can impact the individual's well-being. Inter-individual biological variation and especially age and gender adds a further level of uncertainty. The current standard of comparing test results of an individual to a “population norm” such as population averages can be described as performing to a reasonable level. To address gender and age-associated analyte concentration the standard procedure is to establish normal ranges of an analyte in paediatric and adult populations for females and males but such a coarse method results in information loss (i.e. potential well-being related data) especially for adults, as analytes can show a steady decrease or increase throughout adulthood.

The patient data sets arising from testing require efficient, intricate and robust methods to capture, process and display the data in a consumer-readable format. A further component of the preventative approach and the maintenance of the well-being of an individual is the identification of disease drivers and their control and removal. For example, environmental stressors were estimated to cause over 12 million deaths in 2012 (as set out in the WHO Health Statistics 2016 report). Such environmental stressors are associated with mainly cardiovascular disease (such as stroke and heart disease) and cancer, but also include respiratory diseases and infections, diarrhoeal diseases and neonatal conditions. Amongst the environmental stressors are environmental toxicants such as pesticides, molecular and particulate combustion products, food additives and personal care product components, and a preventative healthcare model requires addressing the problem of environmental toxicants. Current healthcare systems do not address this fundamental disease driver.

Supportive of a preventative approach to healthcare is the concept of biological age (BA) in which an individual's age and health is assessed using biomarker measurements from which an algorithm is constructed to apply a correction to chronological age to derive the individual's BA. The biomarkers used If an individual is found to have a BA which exceeds his/her chronological age corrective measures can be implemented before disease onset. There are several different BA algorithms which are relatively complex, use different biomarkers, partly due to the non-standardised cohorts used, often involve costly genetic analyses of DNA methylation patterns and are not readily interpretable by non-specialists (e.g. WO2015/048665, EP2976433). A proactive, simplistic and consumer-intuitive method of assessing the health of an individual and maintaining the individual's healthspan would support a more effective preventative healthcare model.

### SUMMARY OF THE INVENTION

The invention describes a method of attributing an adjusted chronological age to an individual by measuring one or more analytes in an in vitro biological sample of the individual and comparing the value obtained from the measurement of the analyte to a reference value. Based on the comparison conducted, an adjusted chronological age (ACA) is assigned to the individual which can either be greater than or less than the individual's nominal biological age; alternatively, if there is no difference between the individual's measured analyte values and the reference values then the individual's nominal chronological age is maintained. The analytes used for application of the ACA method are chosen from HbA1c, ASO, CRP, albumin glucose, urea and cystatin C. By assigning an ACA the wellbeing of the individual can be more accurately assessed and interventive measures applied if required. The invention further describes attributing an adjusted chronological age using the aforementioned analytes to biological systems such as defined organs, tissue and physiological systems.

Also described is a microarray comprising binding ligands to the analytes used for chronological age adjustment and a computer program product for use in the invention. The microarray comprises binding ligands, attached to a substrate, to two, three, four, five or six of the following analytes HbA1c, ASO, CRP, albumin, glucose, urea and cystatin C.

The invention further describes a computer program product comprising a computer readable medium having a computer readable code stored thereon, the code being executable by a computer processor to integrate and analyse data comprising measurements of analytes obtained from an individual in the method of invention, to enable allocation of an adjusted chronological age to the individual.

### FIGURES

FIG. 1 Graphical representation of the clinical normal reference range of healthy adults for representative analyte Q.

FIG. 2 ASO concentration median variation by age in healthy adults for two different age stratifications. The lower diagram additionally shows IgG median concentrations.

FIG. 3 Graphical representation of generic ACA application.

Patient 1 has a chronological age corresponding to reference age group A, and has a Z analyte concentration of X which is less than the average concentration for age bracket A and is within the 95% confidence interval reference age of group A; it is also less than the average concentration of reference age group B, which spans the next age range up from age group A. Therefore, patient 1 can be classified has having an increased ACA based on i. having a Z concentration lower than the reference group average ii. having a Z concentration within the lower 95% CI interval of age group A iii. having a Z concentration lower than the reference age group average of age group B. This finding of an increased ACA in a disease-free patient enables a preventative healthcare plan to be implemented.

Patient 2 has a chronological age corresponding to reference age group B, and has a Z analyte concentration represented by the star, which is more than the average concentration for age bracket B and is outside of the upper 95% confidence interval reference age of group B; it is also greater than the average concentration of reference age group B, which spans the previous age range down from age group B. Therefore, patient 2 can be classified has having a decreased ACA based on i. having a Z concentration greater than the reference group average ii. having a Z concentration outside of the upper 95% CI interval of age group B iii. having a Z concentration greater than the reference age group average of age group A.

### DETAILED DESCRIPTION OF THE INVENTION

The invention describes a method of attributing an adjusted chronological age to an individual by measuring an analyte in an in vitro biological sample of the individual and comparing the value obtained from the measurement of the analyte to a reference value. Based on the comparison conducted, an adjusted chronological age (ACA) is assigned to the individual which can either be greater than or less than the individual's nominal chronological age; alternatively, if there is no difference between the individual's measured analyte value and the reference value then the individual's nominal chronological age is maintained.

“Nominal chronological age” is the age provided by the individual, corresponding to the individual's calendar age and can be confirmed using an official record such as a birth certificate. It is preferable that at least two analytes in the individual's in vitro sample are measured. Preferably, the analytes measured are chosen from ASO, HbA1c, CRP, albumin, glucose, urea and cystatin C. The measurement of the concentration of these analytes that vary in individuals categorised as healthy, individuals not displaying or having recently displayed or been diagnosed as diseased, further permits the highlighting of a biological system that may have an increased ACA and efficiently facilitate targeted therapy.

The analytes to be measured are generally assigned to a particular biological system to which they are considered to be most connected to (physiological system, organ, tissue or other recognisable or acknowledged biological unit), hence an adjusted chronological age can equally be assigned to a biological system and can also be termed the biological age of the biological system.

The reference or normal value of an analyte is a statistical measure of central tendency e.g. median, mean value of the analyte, with or without confidence intervals (CI's), interquartile ranges, standard deviations (SD) etc., derived from a population categorised as healthy. The reference value for a specific analyte will normally be derived from data from healthy individuals with a chronological age spanning two or more years e.g. 30-31 years (spans 2 years), 40-42 (spans 3 years) etc. However, it is possible to apply the methods of the invention using a single chronological age healthy reference value for each analyte e.g. a median of 30 years, a mean of 37 years, a median of 42 years (with interquartile range), a mean of 45 years (with 95% CI's or 1×SD) etc; in this instance an ACA for an analyte can be applied by assessing whether the individual's analyte measurement is lower or higher than the single healthy reference value. Age can be represented in days, weeks, months or years. Age stratification can be in days, weeks, months or years, but is preferably in years; the years of a reference age group can span 1, 2, 3, 4, 5, 6, 7, 8, 9, 10, 11, 12, 13, 14, 15, 16, 17, 18, 19, 20, 21, 22, 23, 24, 25, 26, 27, 28, 29, 56, 57, 58, 59, 60, 61, 62, 63, 64, 65, 66, 67, 68, 69, 70, 71, 72 73, 74, 75, 76, 77, 78, 79, 80, 81, 82, 83, 84, 85, 86, 87, 88, 89, 90, 91, 92, 93, 94, 95, 96, 97, 98, 99 or 100 years. Preferably there at least two age groups, more preferably at least three age groups, and as the ACA concept is more suitably applied to adults, the minimum age of a reference age group is usually 17 or 18 years, although lower or higher ages can be used. For a sample cohort age-stratification system for the concentration of an analyte, a uniformly stratified sample cohort takes the form
a to (a+n), (a+n)+1 to (a+2n)+1, (a+2n)+2 to (a+3n)+2, (a+3n)+3 to (a+4n)+3, (a+4n)+4 to (a+5n)+4 . . . etc.
   in which a is the youngest age in years of an individual in the sample cohort and n, the age stratification gap in years, is 1, 2, 3, 4, 5, 6, 7, 8, 9, 10,11, 12, 13, 14, 15, 16, 17, 18, 19, 20, 21, 22, 23, 24, 25, 26, 27, 28, 29, 30.
   e.g. for a=20, n=5 the first three age groups correspond to 20 to 24 years, 25 to 29 years and 30 to 34 years.

The stratification by years (yrs) may also be non-uniform (the age ranges in years differ between two or more groups)

Some examples of age group reference values which can be used are
i. 18 to 44 years, ≥45 years
ii. 18 to 29 years, 30 to 54 years, ≥55 years
iii. 18 to 44 years, 45 to 54 years, ≥55 years
iv. 18 to 29 years, 30 to 44 years, 45 to 54 years, ≥55 years
v. 18 to 29 years, 30 to 44 years, 45 to 54 years, 55 to 64 years, ≥65 years
vi. 18 to 25 years, 26 to 29 years, 30 to 34 years, 35 to 39 years, 40 to 44 years, to 49 years, 50 to 54 years, 55 to 59 years, 60 to 64 years, 65 to 69 years, 70 to 74 years, ≥75 years
vii. 20 to 29 years, 30 to 39 years, 40 to 49 years, 50 to 59 years, 60 to 69 years, to 79 years, 80 to 89 years, 90 to 100 years

For an adjusted chronological age to be attributed to an individual or to a biological system of the individual, the concentration or numerical value measured or assigned to an analyte in a sample taken from the individual chosen from one or more of ASO, urea, CRP, cystatin C, HbA1c, glucose and albumin, must deviate from a reference value, the reference value preferably being from a reference age group derived from healthy individuals incorporating the nominal chronological age of the individual, and either be greater or lower than an average value of the reference group or be numerically closer to a reference value within a different reference age group derived from healthy individuals; it is preferable that at least two different analytes, more preferably at least three or at least four analytes deviate into a different reference age group, the deviation of the analytes being congruent i.e. either indicating a decrease or increase in age. By utilising a multi-analyte approach to derive an ACA reduces the possibility that any deviation from a reference value is due to the individual having naturally lower or higher values of an analyte. Although many analytes in the body are indirectly and/or directly associated with multiple biological systems, many analytes are acknowledged to be predominantly associated with a specific biological system, sometimes with two biological systems and rarely with more than two; the analytes measured in the processes and products of the invention have been characterised by biological system (Table 1), although this does not necessarily exclude them from having an association with other biological systems. Although it is preferable to derive reference values from gender-based, discretely defined geographical cohorts, reference values from combined female/male and/or non-geographically defined cohorts can also be used. To implement the method the healthy population is stratified by age. Any suitable age range can be used for stratification between for example, one to 30 years. A preferred healthy population age stratification is between 5 to 20 years, 5 to 15 or 5 to 10 years. More preferred is a healthy population age stratification of 1 to 5 years, preferably 1, 2, 3, 4 or 5 years. The most preferred healthy population age stratification is 5 years. The method also depends upon the sample analyte(s) used exhibiting a clear trend in concentration with age in a healthy population cohort, whether that be an increase or decrease. It has surprisingly been found that ASO exhibits a significant decrease in concentration with age and is particularly suited to be used in the methods of the invention; HbA1c also exhibits a clear age-related trend in healthy individuals, one that increases with age. The in vitro biological sample from which analyte measurements are taken can generally be any suitable biological sample such as tears, saliva, exhaled breath exudate, interstitial fluid, urine, meconium, sputum, faeces, semen, mucous, lymph, nasal lavage and hair, but is preferably blood and urine. For ASO, HbA1c, CRP, glucose and albumin the preferred sample type is blood, serum or plasma; for urea and cystatin C the preferred sample type is urine.

The method of the invention can be effected at a dedicated wellbeing outlet, hospital or clinic or other location that has the necessary analytical instrumentation to conduct the analyses. Instrumentation can include molecular analyzers using electromagnetic radiation (EMR) and electrical conductance detection technology, mass spectroscopy linked to chromatographic systems, nuclear magnetic resonance based systems or any other suitable analytical instrumentation capable of detecting the listed ACA analytes. By incorporating the analytes to be detected into microarray technology, point of care devices can be used for the analysis thus allowing their application at GP surgeries and residential dwellings. A microarray is small scale technology which enables the analysis of multiple analytes simultaneously using small amounts of sample (EP 0874242). The availability of at-home devices incorporating microarray technology for the ACA biomarkers enables the individual to regularly and conveniently monitor chronological age and therefore react more promptly to an increased ACA.

The invention further describes a microarray comprising binding ligands to two, three, four, five or six of the following analytes HbA1c, ASO, CRP, albumin glucose, urea and cystatin C. Preferably the microarray comprises two, three, four, five or six of the following analytes i. HbA1c and glucose, ii. ASO, iii. CRP iv. Albumin and v. urea and cystatin C, in which only one analyte is selected from i. and v. Preferably, the microarray comprises HbA1c and ASO and optionally albumin and/or CRP.

The invention also describes a computer program product to enable allocation of an adjusted chronological age to the individual. The computer program product comprises a computer readable medium having a stored computer readable code. The code is executable by an in-built computer processor and performs a method for allocating a chronological age to an individual. The method comprises receiving analyte values measured in an in vitro biological sample taken from the individual after which the values are compared to reference values stored in the computer program product, the reference values being from a healthy cohort stratified by age, and based on the comparison effected a chronological age is generated for the individual can be lower, greater (i.e. an ACA) or the same as the individual's nominal chronological age. The generated age is represented on the interfacial screen of the device and be printed as a hardcopy following connection to a printing device. The age can be represented as a specific year, attributed to a year age-range or indicate that the individual has an ACA that is lower or greater than his/her nominal calendar age. The computer program product can also report the concentration values of one or more analytes selected from ASO, HbA1c, glucose, ASO, albumin, urea and cystatin C and/or attribute a specific calendar year age, a year age-range or indicate that the analyte is at a level corresponding to an ACA that is lower or greater than his/her nominal calendar age.

The invention further describes the use of ASO as a biomarker of age of an individual or a biological system such as the immune system; this apparently distinctive ageing biomarker could also be of use in more complex methods to compute biological age such as the Klemera & Doubal algorithm and similar models (Levine 2013). A method of attributing an adjusted chronological age to an individual, is a powerful preventative healthcare process that promotes the maintenance of an individual's well-being and increases healthspan. By measuring an analyte in an in vitro biological sample of the individual and comparing the value obtained from the measurement of the analyte to age-stratified healthy reference values, an adjusted chronological age (ACA) is assigned to the individual which can either be greater than or less than the individual's nominal chronological age; alternatively, if there is no difference between the individual's measured analyte values compared to the reference values used then the individual's nominal chronological age is maintained. It is preferable that at least two analytes in the individual's in vitro sample are measured. By measuring at least two analytes the accuracy of the result is increased i.e. the ascribing of an ACA or maintenance of nominal CA of the individual when two or more analyte concentrations are consistent in their chronological age assignment, may be implemented with greater confidence. Preferably, the analytes measured are chosen from ASO, HbA1c, CRP, albumin, glucose, urea and cystatin C.

Antistreptolysin O (ASO) are autoantibodies to the streptolysin O toxin produced by streptococcal bacteria. Streptococcal bacteria are commonly associated with throat and skin infections and may cause long-term complications such as glomerulonephritis and endocarditis. ASO are predominantly of the IgG1 subclass of IgG immunoglobulins (Falconer 1992).

C-Reactive Protein (CRP) is an acute phase protein produced primarily by the liver. Measurement of CRP is a general marker of inflammation or infection. A high level of CRP in the blood is indicative of an inflammatory process, and can be due to arthritis or inflammatory bowel disease. Elevated CRP may also indicate presence of infection or autoimmune disease. Studies have identified long-term inflammation as a contributing factor to atherosclerosis.

Cystatin C is produced by many cell types. The glomeruli of the kidneys filter cystatin C from the blood at a rate referred to as the glomerular filtration rate (GFR). Cystatin C levels in the blood remain relatively constant when the kidneys are working efficiently. If the kidneys are damaged or diseased, the GFR decreases and cystatin C levels can rise. Elevated cystatin C levels may also be associated with an increased risk of metabolic syndrome, heart disease, heart failure and stroke. Cystatin C levels are also sensitive to changes in thyroid function, and may be lower in hypothyroidism and greater in hyperthyroidism.

Glucose is a simple sugar for which an increased fasting level is indicative of diabetes. Increased levels can be associated with hyperthyroidism, pancreatitis, chronic kidney failure, and rare conditions such as acromegaly and Cushing's syndrome. Various medications such as steroids and diuretics can also increase glucose levels. Decreased levels may be associated with starvation, hypothyroidism (an underactive thyroid gland), extensive liver disease, insulin overdose, and rare conditions such as insulinoma, hypopituitarism and Addison's disease.

HbA1c is formed when haemoglobin in red blood cells (RBCs) combines with glucose in the blood. The HbA1c level is stable, RBCs having a life-time of 2-3 months, providing an accurate long-term index of the average glucose level in the blood. Increased HbA1c levels can be associated with diabetes mellitus, gestational diabetes, acute stress response, corticosteroid therapy and Cushing's syndrome. Urea is a by-product of protein metabolism in the liver. Most urea produced in the liver is removed by the kidneys hence urea levels in the blood are indicative of how well the kidneys are functioning. Elevated urea levels may be due to kidney disease, decreased blood flow to the kidneys, bleeding in the digestive tract, obstruction in the urinary tract or dehydration. Urea levels are high in individuals who consume a high protein diet; low urea levels can occur during pregnancy and chronic liver disease.

### REFERENCES

Falconer A. E. et al. (1992). Distinct IgG1 and IgG3 subclass responses to two streptococcal protein antigens in man: analysis of antibodies to streptolysin O and M protein using standardized subclass-specific enzyme-linked immunosorbent proteins. Immunology, 79: 89-94.
Levine M. E. (2013). Modeling the rate of senescence: can estimated biological age predict mortality more accurately than chronological age? J. Gerontol. A Biol. Sci. Med. Sci, 68(6): 667-674.

### METHODS AND RESULTS

The data in the examples is drawn from patient blood and urinary samples that were collected at Randox Health UK Clinics in Crumlin & Holywood, Liverpool and London. Biochemicals were analysed at Randox Health Holywood, Randox Health London, Randox Health Liverpool and Randox Clinical Laboratory Services in Antrim. Analysers used for measuring biochemicals were Randox Imola, Randox Evolution, Sysmex XS1000i, Roche e801, Roche Urisys 1100 and Siemens Immulite 2000XPi. White blood cells (WBC) and glucose were measured on a Roche Urisys analyzer in a single laboratory (Antrim, NI). Insulin, folic acid, myoglobin and Vitamin B12 were measured on a Roche e801 analyser in the same laboratory (NI). Alanine aminotransferase (ALT), alkaline phosphatase (ALP), aspartate aminotransferase (AST), gamma glutamyltransferase (GGT), creatinine kinase (CK Nac), albumin, ferritn, total bilirubin, total antioxidant status (TAS), pancreatic amylase, lipase, creatinine, cystatin C, rheumatoid factor (RF), calcium, magnesium, sodium, urea, uric acid, C-reactive protein, triglycerides, HDL, HbAc1, cholesterol, immunoglobulin G (IgG), immunoglobulin E (IgE), iron, antistreptolysin O (ASO) and transferrin were measured on a Randox Imola analyser in different laboratories (NI, London & Liverpool). H. pylori was measured on a Siemen's Immulite 2000XPi analyser in a single laboratory (Antrim, NI). Vitamin D, thyroid stimulating hormone, free thyroxine (FT4) and free tri-iodothyronine (FT3) were measured on a Randox Evolution analyser in different laboratories (NI, London & Liverpool). Biochemicals measured are displayed in Table 1. Each analyser is used in its standard manner without modification.

TABLE 1
Analytes measured and assessed for suitability
for use as ACA/biological system biomarkers

| Analyte | Biological System |
|---|---|
| Free thyroxine (FT4) | Thyroid |
| Free tri-iodothyroxine (FT3) | Thyroid |
| Thyroid stimulating hormone (TSH) | Thyroid |
| Glucose | Metabolic syndrome |
| HbA1c | Metabolic syndrome |
| Insulin | Metabolic syndrome |
| C-reactive protein (CRP) | Cardiovascular, infection |
| Creatine kinase | Cardiovascular, muscle |
| Myoglobin | Cardiovascular, muscle, joints |
| Alanine aminotransferase (ALT) | Liver |
| Albumin | General |
| Alkaline phosphatase (ALP) | Liver, bone |
| alpha-1-Antitrypsin (AAT) | Immune |
| Antistreptolysin O (ASO) | Immune, infection |
| Aspartate aminotransferase (AST) | Liver |
| Calcium | Kidney, nutrition, bone |
| Creatinine | Kidney |
| Cystatin C | Kidney |
| Ferritin | Liver, infection |
| Folic acid | Nutrition |
| gamma-Glutamyltransferase (GGT) | Liver |
| H. pylori | Digestive |
| Immunoglobulin E (IgE) | Immune |
| Immunoglobulin G (IgG) | Immune |
| Iron | Nutrition, iron status |
| Lipase | Pancreas |
| Magnesium | Kidney, nutrition |
| Pancreatic amylase | Pancreas |
| Rheumatoid factor (RF) | Immune, infection |
| Sodium | Kidney |
| Total antioxidant status (TAS) | Nutritional |
| Bilrubin | Liver |
| Transferrin | Iron status |
| Urea | Kidney |
| Uric acid | Kidney |
| Vitamin B12 | Nutrition |
| Vitamin D | Bone |
| White blood cell count (WBC) | Immune |

Criteria for consideration as a biomarker for ACA inclusion was based on age-related concentration changes in a healthy population (n>4,000) supported by statistically significant differences between different age ranges and correlation statistics of pairs of analytes. The analytes, HbA1c, glucose, ASO, CRP, albumin, urea and cystatin C qualified for use as biomarkers of ACA and biological systems. The preferred grouping based on greatest statistical power were ASO, HbA1c, cystatin C, CRP and albumin.

The use of ASO as a biomarker for the ageing of biological systems and individuals has not been previously disclosed. This discovery, and the discrete age-related values found would enable ASO concentration to be used in more complex algorithms for computing the biological age of an individual or a biological system, particularly the immune system.

TABLE 2a
Median analyte concentration of healthy females resident in UK. Analytes
categorised according to recognised system/organ association. Kruskall-Wallis statistic for
each analyte P < 0.0001.

| | | | | General | | | |
|---|---|---|---|---|---|---|---|
| | Inflammatory | Dietary | | systemic | Immune | Kidney | |
| Age | CRP | HbA1c | Glucose | Albumin | ASO | Urea | Cys C |
| bracket | mg/l | mm/mol | mmol/l | g/l | IU/ml | mmol/l | mg/l |
| 18-24 | 1.30 | 29.85 | 4.82 | 45.25 | 198.00 | 4.10 | 0.66 |
| 25-29 | 1.69 | 29.90 | 4.79 | 44.57 | 217.00 | 4.18 | 0.65 |
| 30-34 | 1.40 | 29.81 | 4.72 | 44.80 | 148.00 | 4.29 | 0.68 |
| 35-39 | 1.20 | 30.02 | 4.78 | 44.70 | 149.00 | 4.35 | 0.66 |
| 40-44 | 1.20 | 30.98 | 4.88 | 44.47 | 146.00 | 4.57 | 0.67 |
| 45-49 | 1.20 | 32.13 | 4.94 | 44.00 | 119.00 | 4.59 | 0.68 |
| 50-54 | 1.40 | 31.79 | 5.00 | 44.00 | 91.00 | 5.00 | 0.70 |
| 55-59 | 1.70 | 34.07 | 5.10 | 44.10 | 97.00 | 5.38 | 0.75 |
| 60-64 | 1.60 | 34.19 | 5.16 | 44.20 | 82.40 | 5.50 | 0.77 |
| 65-69 | 1.40 | 34.37 | 5.18 | 43.51 | 68.10 | 5.51 | 0.81 |
| 70-74 | 1.60 | 35.92 | 5.40 | 43.75 | 60.60 | 6.2 | 0.90 |
| ≥75 | | | | | 45.00 | | 0.91 |

TABLE 2b
Median analyte concentration of healthy males resident in UK. Analytes
categorised according to recognised system/organ association. Kruskall-Wallis statistic for
each analyte P < 0.0001.

| | | | | General | | | |
|---|---|---|---|---|---|---|---|
| | Inflammatory | Dietary | | systemic | Immune | Kidney | |
| Age | CRP | HbA1c | Glucose | Albumin | ASO | Urea | Cys C |
| bracket | mg/l | mm/mol | mmol/l | g/l | IU/ml | mmol/l | mg/l |
| 18-24 | 0.90 | 28.12 | 5.00 | 46.90 | 244.00 | 5.42 | 0.74 |
| 25-29 | 0.90 | 28.26 | 4.92 | 46.90 | 213.00 | 5.56 | 0.72 |
| 30-34 | 1.20 | 29.05 | 5.03 | 46.50 | 170.00 | 5.25 | 0.72 |
| 35-39 | 1.08 | 30.39 | 5.11 | 46.20 | 167.00 | 5.46 | 0.72 |
| 40-44 | 1.10 | 30.37 | 5.20 | 46.00 | 152.00 | 5.46 | 0.73 |
| 45-49 | 1.17 | 31.63 | 5.26 | 45.60 | 125.00 | 5.55 | 0.74 |
| 50-54 | 1.20 | 32.00 | 5.30 | 45.12 | 101.00 | 5.60 | 0.77 |
| 55-59 | 1.30 | 32.76 | 5.37 | 45.00 | 100.00 | 5.61 | 0.78 |
| 60-64 | 1.30 | 33.55 | 5.45 | 44.90 | 91.00 | 5.80 | 0.79 |
| 65-69 | 1.50 | 34.09 | 5.41 | 44.20 | 85.00 | 5.77 | 0.81 |
| 70-74 | 1.75 | 36.17 | 5.47 | 44.60 | 52.00 | 6.48 | 0.92 |
| ≥75 | | | | | 49.00 | | 1.07 |

TABLE 2c
Median analyte concentration of healthy females (F) & males (M) resident in UK
(n > 4,000). Analytes were categorised according to recognised system/organ association.
For both male and female, the analyte concentration between the three age groups was
statistically significant for every analyte.

| | | | | General | | | |
|---|---|---|---|---|---|---|---|
| | Inflammatory | Dietary | | systemic | Immune | Kidney | |
| Age | CRP | HbA1c | Glucose | Albumin | ASO | Urea | Cys C |
| bracket | mg/l | mm/mol | mmol/l | g/l | IU/ml | mmol/l | mg/l |
| F 18-44 | 1.23 | 30.28 | 4.80 | 44.60 | 151.60 | 4.31 | 0.66 |
| F 45-54 | 1.31 | 31.90 | 4.98 | 44.00 | 105.70 | 4.74 | 0.69 |
| F ≥55 | 1.59 | 34.32 | 5.13 | 43.80 | 81.00 | 5.38 | 0.78 |
| M 18-44 | 1.00 | 29.14 | 5.01 | 46.60 | 173.80 | 5.29 | 0.72 |
| M 45-54 | 1.10 | 31.61 | 5.27 | 45.50 | 120.00 | 5.57 | 0.75 |
| M ≥55 | 1.39 | 33.80 | 5.40 | 44.60 | 91.10 | 5.73 | 0.80 |

### Applying ACA

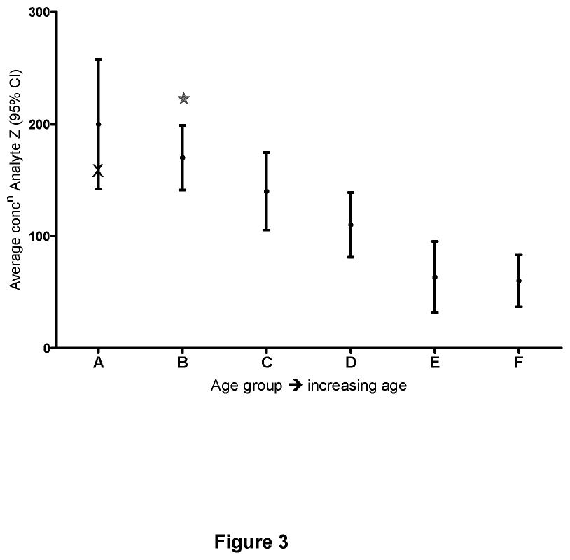

The ACA method identifies the analyte median, or mean concentration (or other measure of statistical central tendency) of a designated healthy age group which incorporates the individual's chronological age. If the individual's analyte concentration is less than this average value then a reduced ACA can be assigned to the individual. Alternatively, a reduced ACA can be assigned if the individual's analyte concentration is lower than the 75% or 95% confidence intervals (or other suitable CI's) of the average analyte concentration of the age group; a further alternative is to assign a reduced ACA if the individual's analyte concentration is equal to or less than the average analyte concentration of the preceding age group or the age group preceding that; another alternative is to assign a reduced ACA if the individual's analyte concentration is within the 75% or 95% confidence intervals of the average analyte concentration of the preceding age group as long as these values do not equal or exceed the average analyte concentration of the individual's chronological age group. For an increased ACA, the opposite adjectival descriptors of ACA and analyte concentration used in the above method are applied, with ‘more’ replacing ‘less’, ‘greater’ replacing ‘lower’, ‘increased’ replacing ‘reduced’, ‘are less than’ replacing ‘exceed’ etc. FIG. 3 pictorially exemplifies the ACA method. The methods can be applied using both uniform or non-uniform stratified sample age-groups.

The methods can be applied to one or more of each of ASO, HbA1c, glucose, CRP, albumin, urea and cystatin C to assign either an ACA to the individual or to a biological system. Preferably, for an adjusted chronological age to be applied to an individual or to a biological system, each of two analytes will exhibit either concentration reductions or increases relative to their respective healthy values; more preferably, each of three or more analytes will exhibit either concentration reductions or increases relative to their respective healthy values. Individuals with an increased ACA for some analytes and a decreased ACA for others, an ACA can be assigned on a biological system basis. Table 3 lists several examples. The analyte ASO is a particularly strong indicator of chronological age and is therefore more likely to be used as a single biomarker for deriving an ACA for an individual or the immune system of an individual.

TABLE 3
Analyte profiles of healthy patients whose profiles
indicate either a lower or higher adjusted chronological
age. Each had CRP values < 2.00 ng/ml.

| | | Dietary | General systemic | Immune |
|---|---|---|---|---|
| | | HbA1c | Albumin | ASO |
| Patient | Age | mm/mol | g/l | IU/ml |
| | female | | | |
| 1 | 50 | 30.25 | 48.19 | 104 |
| 2 | 48 | 30.64 | 44.91 | 139.6 |
| 3 | 42 | 29.02 | 42.30 | 151.3 |
| 4 | 43 | 31.23 | 45.80 | 158.6 |
| 5 | 48 | 41.5 | 44.40 | 77.0 |
| 6 | 44 | 36.49 | 41.40 | 48.4 |
| | male | | | |
| 7 | 52 | 33.78 | 43.70 | 54.3 |
| 8 | 48 | 32.61 | 43.60 | 91.6 |
| 9 | 42 | 35.66 | 47.30 | 83.4 |
| 10 | 52 | 29.26 | 47.00 | 126.8 |
| 11 | 45 | 29.24 | 47.70 | 153.4 |
| 12 | 41 | 28.21 | 48.30 | 212.3 |

Patient 2, chronological age 48, using the healthy age group median values of age groups of Table 2a as reference placing patient 2 in the 45 to 49 age group, had levels of ASO, HbA1c, and albumin levels all corresponding to an age cohort lower than 45 to 49 years and hence would be afforded a reduced ACA. As the adjusted ACA for some of the analytes belong to different age brackets, it is preferable that the individual is ascribed a decreased ACA; if the analytes show uniformity in the adjusted ACA, which could 2, 3 or more analytes, then the ACA could be reported as a specific age bracket. Furthermore, a reduced ACA can be ascribed to the immune and dietary biological systems. Using the age group analyte stratification of Table 2c placing patient 2 in the 45 to 54 age group and applying the 75% confidence intervals of the mean values as the measure of central tendency for each age group, patient 2 can be assigned an ACA of 18-44, and likewise her immune and dietary systems.

Patient 5, chronological age 48, using the healthy age group median values of age groups of Table 2a as reference placing patient 5 in the 45 to 49 age group, had levels of ASO, HbA1c, and albumin all corresponding to an age cohort higher than 45 to 49 years and hence would be afforded an increased ACA. As the adjusted ACA for some of the analytes belong to different higher age brackets, it is preferable that the individual is ascribed an increased ACA; if the analytes show uniformity in the adjusted ACA, which could be 2, 3 or more analytes, then the ACA could be reported as a specific age bracket. Furthermore, an increased ACA can be ascribed to the immune and dietary biological systems. Importantly, this individual has levels of analytes that although non-pathological, indicate that they are closer to a disease state than indicated by their nominal chronological age; by highlighting an increased ACA the individual can redress the situation through behavioural and/or therapeutic management, and delay possible disease onset. For example, as the HbA1c level, a marker associated with metabolic syndrome/diabetes, is higher than expected, the individual could be, for example, encouraged to adapt their diet, increase exercise and to regularly monitor HbA1c levels. The lower than expected level of ASO, an immune system marker, could encourage immune system boosting intervention such as plasma therapy, immunoglobulin therapy, interferon-gamma therapy, growth factor therapy and stem cell therapy.

Patient 10, chronological age 52, and using the healthy age group median values of age groups of Table 2b as reference thus placing patient 10 in the 50 to 54 age group, had levels of ASO, HbA1c and albumin all corresponding to an age cohort lower than 45 to 49 years and hence would be afforded a decreased ACA. As the adjusted ACA for some of the analytes belong to different age brackets, it is preferable that the individual is ascribed a decreased ACA; if the analytes show uniformity in the adjusted ACA, which could be 2, 3 or more analytes, then the ACA could be reported as a specific age bracket. Furthermore, a reduced ACA can be ascribed to the immune and dietary biological systems. Using the age group analyte stratification of Table 2c placing patient 10 in the 45 to 54 age group, and applying the 75% confidence intervals of the mean values as the measure of central tendency for each age group, patient 10 can be assigned an ACA of 18-44, and likewise his dietary system.

The data tables, figures and patient examples show that the different age group stratifications provide a similar ACA result, either an increase or decrease. Different age group stratifications can provide different levels of detail, a finer scale age group stratification often providing greater detail than a coarser scale age group stratification e.g. Tables 2a/2b (finer scale) vs Table 2c (coarser scale) and exemplified by patient 10.

Correlation coefficient analyses of the 8 analytes indicate that glucose and HbA1c, urea and cystatin C and cystatin C and CRP show significant and consistent correlation across various age groups for each gender. Tables 5 and 6 are representative examples for males and females in the age cohorts 45-49 years and 50-54 years.

TABLE 5a
Males aged 45-49 years correlation matrix correlation coefficients;

| | albumin | ASO | CRP | cystatin C | glucose | HbA1c | urea |
|---|---|---|---|---|---|---|---|
| albumin | | 0.023 | −0.001 | −0.123* | 0.032 | 0.023 | −0.051 |
| ASO | 0.023 | | 0.029 | −0.046 | 0.135* | 0.107 | 0.002 |
| CRP | −0.001 | 0.029 | | 0.281* | −0.008 | 0.015 | −0.082 |
| cystatin C | −0.123* | −0.046 | 0.281* | | −0.053 | 0.047 | 0.163* |
| glucose | 0.032 | 0.135* | −0.008 | −0.053 | | 0.257* | −0.024 |
| HbA1c | 0.023 | 0.107 | 0.015 | 0.047 | 0.257* | | −0.048 |
| urea | −0.051 | 0.002 | −0.082 | 0.163* | −0.024 | −0.048 | |

*P < 0.05

TABLE 5b
Males aged 50-54 years correlation matrix correlation coefficients;

| | albumin | ASO | CRP | cystatin C | glucose | HbA1c | urea |
|---|---|---|---|---|---|---|---|
| albumin | | −0.068 | −0.111 | −0.113 | 0.041 | −0.003 | −0.034 |
| ASO | −0.068 | | 0.106 | −0.016 | 0.110 | 0.022 | −0.059 |
| CRP | −0.113 | 0.106 | | 0.138* | 0.094 | 0.001 | −0.063 |
| cystatin C | −0.113 | −0.016 | 0.138* | | −0.011 | −0.079 | 0.257* |
| glucose | 0.041 | 0.110 | 0.094 | −0.011 | | 0.412* | 0.033 |
| HbA1c | −0.003 | 0.022 | 0.001 | −0.079 | 0.412* | | −0.015 |
| urea | −0.034 | −0.059 | −0.063 | 0.257* | 0.033 | −0.015 | |

*P < 0.05

TABLE 6a
Females aged 45-49 years correlation matrix correlation coefficients;

| | albumin | ASO | CRP | cystatin C | glucose | HbA1c | urea |
|---|---|---|---|---|---|---|---|
| albumin | | −0.001 | −0.119* | −0.051 | 0.127* | 0.002 | 0.029 |
| ASO | −0.001 | | −0.041 | −0.056 | −0.009 | −0.113 | 0.026 |
| CRP | −0.119* | −0.041 | | 0.130* | 0.073 | 0.063 | −0.014 |
| cystatin C | −0.051 | −0.056 | 0.130* | | 0.037 | −0.058 | 0.313* |
| glucose | 0.127* | −0.009 | 0.073 | 0.037 | | 0.124* | 0.086 |
| HbA1c | 0.002 | −0.113 | 0.063 | −0.058 | 0.124* | | 0.058 |
| urea | 0.029 | 0.026 | −0.014 | 0.313* | 0.086 | 0.058 | |

*P < 0.05

TABLE 6b
Females aged 50-54 years correlation matrix correlation coefficients;

| | albumin | ASO | CRP | cystatin C | glucose | HbA1c | urea |
|---|---|---|---|---|---|---|---|
| albumin | | −0.088 | −0.081 | −0.091 | 0.055 | −0.022 | −0.032 |
| ASO | −0.088 | | 0.075 | 0.036 | −0.049 | −0.162* | −0.014 |
| CRP | −0.081 | 0.075 | | 0.109 | 0.091 | 0.077 | −0.072 |
| cystatin C | −0.091 | 0.036 | 0.109 | | 0.063 | 0.032 | 0.198* |
| glucose | 0.055 | −0.049 | 0.091 | 0.063 | | 0.092 | 0.121* |
| HbA1c | −0.022 | −0.162* | 0.077 | 0.032 | 0.092 | | 0.033 |
| urea | −0.032 | −0.014 | −0.072 | 0.198* | 0.121* | 0.033 | |

*P < 0.05

TABLE 7a
Comparisons for ASO for both females and males
applied across various age groups (Kruskal Wallis
statistic P < 0.0001 for each gender).

| Dunn's Multiple | Females significant | Males Significant |
|---|---|---|
| Comparison Test | P < 0.05 | P < 0.05 |
| >=75 vs 70-74 | No | No |
| >=75 vs 65-69 | No | No |
| >=75 vs 60-64 | No | No |
| >=75 vs 55-59 | No | No |
| >=75 vs 50-54 | No | Yes |
| >=75 vs 45-49 | Yes | Yes |
| >=75 vs 40-44 | Yes | Yes |
| >=75 vs 35-39 | Yes | Yes |
| >=75 vs 30-34 | Yes | Yes |
| >=75 vs 25-29 | Yes | Yes |
| >=75 vs 18-24 | Yes | Yes |
| 70-74 vs 65-69 | No | No |
| 70-74 vs 60-64 | No | No |
| 70-74 vs 55-59 | No | No |
| 70-74 vs 50-54 | No | No |
| 70-74 vs 45-49 | No | Yes |
| 70-74 vs 40-44 | Yes | Yes |
| 70-74 vs 35-39 | Yes | Yes |
| 70-74 vs 30-34 | Yes | Yes |
| 70-74 vs 25-29 | Yes | Yes |
| 70-74 vs 18-24 | Yes | Yes |
| 65-69 vs 60-64 | No | No |
| 65-69 vs 55-59 | No | No |
| 65-69 vs 50-54 | No | No |
| 65-69 vs 45-49 | Yes | Yes |
| 65-69 vs 40-44 | Yes | Yes |
| 65-69 vs 35-39 | Yes | Yes |
| 65-69 vs 30-34 | Yes | Yes |
| 65-69 vs 25-29 | Yes | Yes |
| 65-69 vs 18-24 | Yes | Yes |
| 60-64 vs 55-59 | No | No |
| 60-64 vs 50-54 | No | No |
| 60-64 vs 45-49 | Yes | No |
| 60-64 vs 40-44 | Yes | Yes |
| 60-64 vs 35-39 | Yes | Yes |
| 60-64 vs 30-34 | Yes | Yes |
| 60-64 vs 25-29 | Yes | Yes |
| 60-64 vs 18-24 | Yes | Yes |
| 55-59 vs 50-54 | No | No |
| 55-59 vs 45-49 | No | No |
| 55-59 vs 40-44 | Yes | Yes |
| 55-59 vs 35-39 | Yes | Yes |
| 55-59 vs 30-34 | Yes | Yes |
| 55-59 vs 25-29 | Yes | Yes |
| 55-59 vs 18-24 | Yes | Yes |
| 50-54 vs 45-49 | No | No |
| 50-54 vs 40-44 | Yes | Yes |
| 50-54 vs 35-39 | Yes | Yes |
| 50-54 vs 30-34 | Yes | Yes |
| 50-54 vs 25-29 | Yes | Yes |
| 50-54 vs 18-24 | Yes | Yes |
| 45-49 vs 40-44 | No | Yes |
| 45-49 vs 35-39 | No | Yes |
| 45-49 vs 30-34 | No | Yes |
| 45-49 vs 25-29 | Yes | Yes |
| 45-49 vs 18-24 | Yes | Yes |
| 40-44 vs 35-39 | No | No |
| 40-44 vs 30-34 | No | No |
| 40-44 vs 25-29 | No | No |
| 40-44 vs 18-24 | No | Yes |
| 35-39 vs 30-34 | No | No |
| 35-39 vs 25-29 | No | No |
| 35-39 vs 18-24 | No | No |
| 30-34 vs 25-29 | No | No |
| 30-34 vs 18-24 | No | No |
| 25-29 vs 18-24 | No | No |

TABLE 7b
Comparisons for ASO for females and males across four
age groups (Kruskal Wallis P < 0.0001 for each gender)

| | | | Medians | | |
|---|---|---|---|---|---|
| Dunn's Multiple | Females | Males | IU/ml | | |
| Comparison Test | KW statistic = 139.2 | KW statistic = 227.2 | Age | Female | Male |
| ≥60 vs 45-59 | *** | *** | 18-29 | 193 | 225 |
| ≥60 vs 30-44 | *** | *** | 30-44 | 144 | 163 |
| ≥60 vs 18-29 | *** | *** | 45-59 | 103 | 116 |
| 45-59 vs 30-44 | *** | *** | ≥60 | 70 | 82 |
| 45-59 vs 18-29 | *** | *** | | | |
| 30-44 vs 18-29 | * | *** | | | |

TABLE 7c
Comparisons for ASO for females and males across three
age groups (Kruskal Wallis P < 0.0001 for each gender).

| Dunn's Multiple | Females | Males |
|---|---|---|
| Comparison Test | KW statistic = 130.60 | KW statistic = 211.0 |
| ≥60 vs 45-59 | *** | *** |
| ≥60 vs 18-44 | *** | *** |
| 45-59 vs 18-44 | *** | *** |

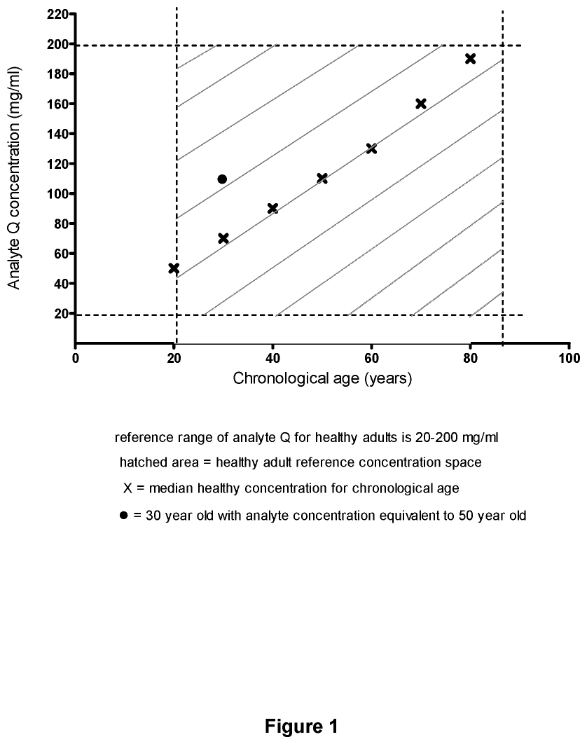

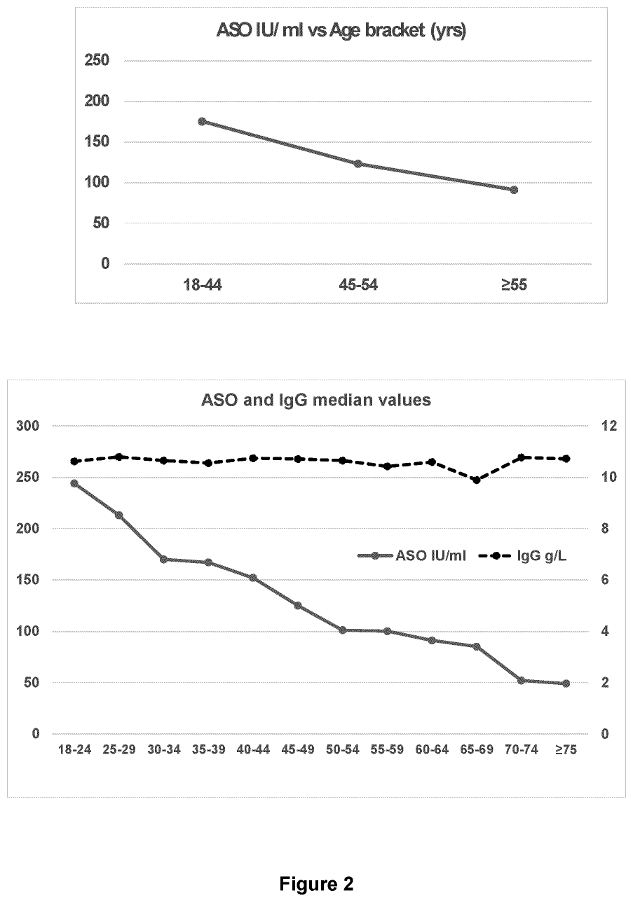

An advantage of the ACA method for an individual classified as healthy, but who has an analyte concentration corresponding to a higher age bracket (increased ACA) is the highlighting of a possible pre-disease state, enabling preventative measures, whether behavioural or medical, to be initiated. FIG. 1 provides a graphical representation of how further refining the detail of clinically-applied healthy adult analyte normal ranges using ACA can support preventative healthcare. For example, an individual who has a higher ACA than his nominal chronological age would be informed that an increased ACA has been discovered. The individual would be told of the analyte(s) involved and of possible interventions to remedy the increased ACA. This enables a preventative approach to disease management. The steady depletion of ASO from 18 to 90 years in both males and females (FIG. 2) makes ASO a particularly strong marker for use in ACA analysis in individuals and for the immune system.

1. A method of attributing an adjusted chronological age to an individual and/or to a biological system of the individual comprising
measuring one or more analyte selected from the group consisting of antistreptolysin (ASO), glycated haemoglobin (HbA1c), cystatin C, albumin and C reactive protein (CRP) in an in vitro biological sample of the individual by contacting a microarray comprising two or more binding ligands that bind to the one or more analytes with the biological sample;
comparing the measured value obtained from the measurement of the analyte to one or more reference values of the analyte from a healthy population; and
based on the comparison either attribute an adjusted chronological age to the individual and/or a biological system of the individual or maintain the chronological age and/or a biological system of the individual.

2. The method of claim 1 in which two or more analytes are measured within the sample and in which at least two analyte measurements indicate that the individual or a biological system of the individual has either a decreased chronological age or an increased chronological age.

3. (canceled)

4. The method of claim 1, in which the analytes measured are ASO and HbA1c.

5. The method of claim 1, in which the measurement value of ASO in the individual which is lower than a healthy reference value of ASO indicates an increased chronological age and the measurement value of HbA1c in the individual which is higher than a healthy reference value of HbA1c indicates an increased chronological age.

6. The method of claim 1, in which the healthy population reference values are in age stratified groups, preferably of from one to fifty years.

7. The method of claim 6 in which the healthy population age stratification consists of 2 to 50 age groups.

8. The method of claim 7 in which the healthy population age stratification consists of three, four, five, six, seven, eight, nine, ten, 11, 12, 13, 14 or age groups.

9. A microarray comprising binding ligands to two, three, four or five of the following analytes
i) ASO
ii) HbA1c and glucose
iii) CRP
iv) albumin
v) cystatin C and urea
in which only one analyte is selected from ii) and v).

10. The microarray of claim 9 which comprises ASO and HbA1c.

11. The microarray of claim 10 which further comprises CRP and/or cystatin C.

12. The method of claim 1, wherein the microarray comprises binding ligands to two, three, four or five of the following analytes
i) ASO
ii) HbA1c and glucose
iii) CRP
iv) albumin
v) cystatin C and urea
in which only one analyte is selected from ii) and v).

13. A computer program product comprising a computer readable medium having a computer readable code stored thereon, the code being executable by a computer processor to perform a method for allocating an adjusted chronological age to an individual and/or a biological system, the method comprising
(i) receiving an analyte value measured in an in vitro biological sample taken from the individual;
(ii) comparing the analyte value measured in (i) to one or more reference values from a healthy population stored in the computer program product;
(iii) based on the comparison effected in (ii), a generated age of the individual and/or biological system which is lower or greater than the individual's nominal chronological age, results in an adjusted chronological age for the individual and/or the biological system being generated
(iv) preparing a report that includes a reference to the generated adjusted chronological age.

14. The computer program product of claim 13 which further comprises within a report a reference as to whether one or more analytes selected from ASO, HbA1c, CRP, albumin, glucose, urea and cystatin C are each at a concentration level which is lower or greater than the concentration level of that of a healthy population reference value.

15. The computer program product of claim 14 in which the reference value is derived from a healthy cohort of a similar age and sex to the individual, and preferably from a similar geographical demographic as the individual.

16. A method for attributing an adjusted chronological age to an individual or a biological system comprising measuring one or more of antistreptolysin (ASO), glycated haemoglobin (HbA1c), cystatin C, and C reactive protein (CRP).

17. The method of claim 16, wherein the method measures ASO in an in vitro blood sample.
